(19) United States
(12) Patent Application Publication
(10) Pub. No.: US 2026/0325149 A1
(43) Pub. Date: Oct. 22, 2026

(54) STERILE FIELD CLOT CATCHER DEVICE, MODULE AND METHODS

(71) Applicant: Von Vascular, Inc., Weston, FL (US)

(72) Inventors: Victor GAMEZ, Fort Lauderdale, FL (US); Manning J. HANSER, Weston, FL (US); Alfonso HERMIDA, Miramar, FL (US)

(73) Assignee: Von Vascular, Inc., Weston, FL (US)

(21) Appl. No.: 19/669,273

(22) Filed: May 6, 2026

Related U.S. Application Data
(63) Continuation of application No. 18/373,955, filed on Sep. 27, 2023, now Pat. No. 12,642,543.
Application No. 18/373,955 is a continuation-in-part of application No. 18/123,973, filed on Mar. 20, 2023, now Pat. No. 12,642,542.
(60) Provisional application No. 63/321,706, filed on Mar. 20, 2022.
(60) Provisional application No. 63/325,778, filed on Mar. 31, 2022.
(60) Provisional application No. 63/335,168, filed on Apr. 26, 2022.
(60) Provisional application No. 63/474,167, filed on Jul. 26, 2022.

(51) Int. Cl. A61B 17/22; A61B 17/00

(52) U.S. Cl. CPC A61B 17/22; A61B 2017/00292; A61B 2017/22079

(57) ABSTRACT

A clot capture device assists a surgeon or medical technician to directly see a degree of blood displacement and what type of clot is being or has been extracted, and preferably, all on the sterile field at convenient reach and visual observation by the user (physician or technician), and without cessation of a clot removal procedure. Aspirated blood and a retrieved clot or clots are ejected and deposited on a generally horizontally oriented porous filter and/or foraminous strainer in a co-compartment, for separation thereof. The clot or clots stay in the co-compartment atop the strainer/filter, while extracted blood drains into a blood collection compartment below.

### CROSS REFERENCE TO RELATED APPLICATIONS

This application is a continuation of U.S. patent application Ser. No. 18/373,955, entitled “Sterile Field Clot Catcher Device, Module and Methods,” filed Sep. 27, 2023, which is a continuation-in-part of U.S. patent application Ser. No. 18/123,973, entitled “Adaptive Pressure-Cyclical Aspiration Device and Methods,” filed Mar. 20, 2023, which claims priority to and the filing date benefit of U.S. Provisional Patent Application Nos. 63/474,167, filed Jul. 26, 2022; U.S. Provisional Patent Application No. 63/335,168, filed Apr. 26, 2022; U.S. Provisional Patent Application No. 63/325,778, filed Mar. 31, 2022; and U.S. Provisional Application No. 63/321,706, filed Mar. 20, 2022.

### BACKGROUND OF THE INVENTION

The present disclosures relate, inter alia, to improved acute ischemic event therapies, and equipment and methods for implementing same to effectively and efficiently remove a clot occluding a vessel in a facilitated manner. And more particularly, to methods and devices which advantageously permit examination and identification analysis of a nature of a retrieved clot while remaining on a sterile field without requiring cessation of an extraction process.

The current disclosures relate generally to a system that employs thrombotic extraction strategies for removing a clot occluding a blood vessel with applied aspiration via an inserted clot-extracting catheter in an occluded vessel.

Heretofore, conventional clot aspiration systems typically have clot retrieval/collection devices disposed in-line with the suction path. This requires the user to stop the suction pump in order to retrieve the clot. Such approaches by other systems means that clot retrieval from the clot catcher for observation, examination and identification requires a stoppage of the clot removal aspiration process. This results in critical delay in time sensitive therapeutic procedures often to the detriment of the patient being treated.

It would therefore be highly desirable to create a system and method which can be effective to allow a physician and/or qualified technician to retrieve the extracted clot or clots without having to halt the aspiration process, and which broadly addresses and overcomes these and other drawbacks of prior art approaches.

### SUMMARY OF THE INVENTION

In accordance with these and other objects of the invention, there is provided a thrombotic extraction system which advantageously includes a method and structural adaptation, in a form, for example, which is embodied to include an independent device or integrated module that permits examination of an extracted blood clot or clots without requiring a pause in the treatment process while advantageously remaining on the sterile field.

In accordance with an advantageous embodiment, a clot catcher (or alternatively referred to herein as a “clot collector”) is built into an advantageously sterile blood collection canister or collection compartment in order to help the surgeon or medical technician to directly see a degree of blood displacement and what type of clot is being or has been extracted. More preferably, all on the sterile field at convenient reach and visual observation by the user (physician or technician).

In accordance with an embodiment of the invention, aspirated blood and a retrieved clot or clots are ejected and deposited on a porous filter and/or foraminous strainer in a co-compartment, for separation thereof. The clot or clots stay in the co-compartment atop the strainer/filter, while extracted blood drains into a reservoir/canister/compartment below. According to an advantageous embodiment, the reservoir/canister/compartment is sterile.

According to a particularly advantageous embodiment, the reservoir/canister/ compartment, referred to herein generically as a “blood collection chamber,” can hold over 1000 cc of extracted blood on the sterile field.

A clot capture device (clot catcher) is advantageously fully integrated with a blood collection pump and control assembly comprising an aspiration pump and also optionally various controls, that allows the capture and immediate removal of the aspired clot without having to exit the sterile field or halt a collection process.

The clot catcher optionally includes a lid over the co-compartment disposed above the strainer, conveniently provided in a form of a transparent window, that can be removed or opened for collection of the extracted clot for subsequent examination and analysis. The clot catcher co-compartment bottom includes a perforated strainer provided for example in a form of a sieve or porous filter, etc., referred to generically herein by the term “strainer” in order to allow the passage of blood to a downstream side thereof while retaining the extracted clot on an upstream side above the filter/strainer.

An integrated extraction pump can also be optionally operated with the cover of the clot catcher completely removed or in an open state for even faster and easier clot removal by the user.

While several embodiments of the present disclosure are described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, Within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. The terms and expressions which have been employed herein are used as terms of description and not of limitation, and there is no intention, in the use of such terms and expressions, of excluding any equivalents of the features shown and described (or portions thereof), and it is recognized that various medications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention.

Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method.

Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method.

Additionally, the order in which a particular method occurs may or may not strictly adhere to the order of the corresponding steps or methodologies shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be personnel in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all medications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e. g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Various preferred embodiments are described herein with references to the drawings in which merely illustrative views are offered for consideration, whereby:

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

The above, and other objects, features and advantages of the present invention will become apparent from the following description read in conjunction with the accompanying drawings.

Features, aspects, and advantages are described below with reference to the drawings, which are intended to illustrate, but not to limit, the invention. In the drawings, like reference characters denote corresponding features consistently throughout similar embodiments.

Corresponding reference characters indicate corresponding components throughout the several views of the drawing. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention.

Also, common but well-understood elements that are useful or necessary in a commercially feasible embodiment are often not depicted in order to facilitate a less obstructed view of these various embodiments of the present invention.

### BRIEF DESCRIPTION OF THE DRAWINGS

FIG. 1A is a perspective view depicting an example of a pump system incorporating a clot catcher as an integral sub-component module in accordance with an embodiment of the invention;

FIG. 1B is a top plan view of the pump system including the clot catcher of the embodiment of FIG. 1A;

FIG. 2 depicts a schematic representation of core components of a system according to an embodiment of the present invention in which the clot catcher is located separate from a pump and control system;

FIG. 3 is a perspective view of an example of a pump system connected to a clot catcher which is located separate from a pump and control system as schematically represented in FIG. 2 according to an embodiment of the invention;

FIG. 4A is a side perspective exemplary view of a pump system in general accordance with FIGS. 1A and 1B being shown with a top housing portion removed exposing the integrated clot catcher blood collection compartment; and

FIG. 4B is a top perspective view of the pump system of FIG. 4A.

### DETAILED DESCRIPTION OF THE PREFERRED EMBODIMENT

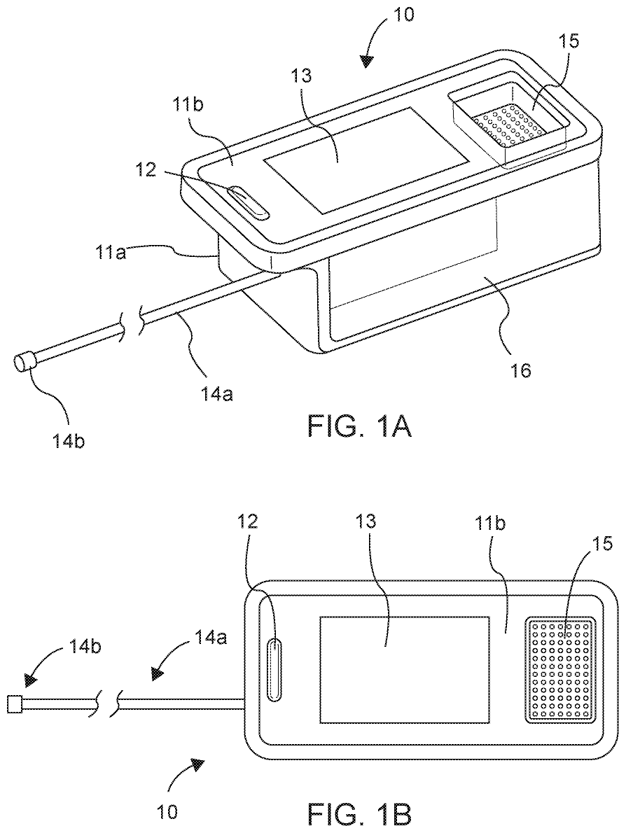

Referring now to the figures, a pump system incorporating a clot catcher as an integral sub-component/module in accordance with an embodiment of the invention is depicted generally at 10 in FIGS. 1A and 1B.

Pump system 10 according to the exemplary embodiment depicted in FIGS. 1A and 1B optionally includes a housing comprised of a housing bottom 11a and a housing top 11b. A control button or switch 12 is advantageously provided to permit convenient access by the operator/user to optionally selectively control an operating mode of pump system 10. A touch screen 13 is also optionally provided to control various operational aspects of the pump system 10.

Connected to the pump system is a conduit for connecting to an operational catheter, and which includes a length of suction tubing 14a having an aspiration catheter connector 14b at or proximate to a terminal end thereof.

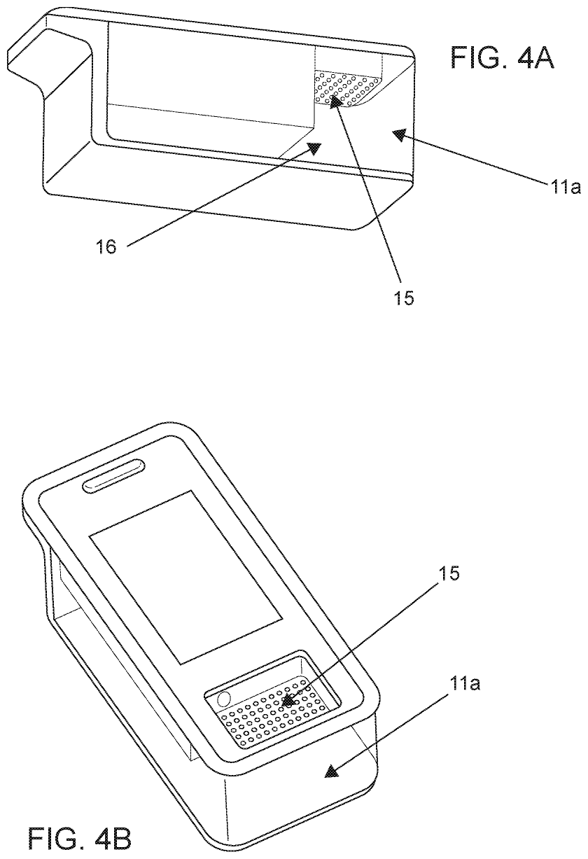

In the depicted embodiment, a clot catcher 15 according to an embodiment of the invention is provided advantageously in a form of an integrally incorporated sub-component of pump system 10. A blood collection chamber 16 is provided below a porous filter/strainer (not shown in FIGS. 1A and 1B, but can be best seen for example in FIGS. 4A and 4B). A transparent lid closure atop clot catcher 15 is optionally provided (not shown) and is advantageously openable or removable to facilitate removal of a collected clot or clots for detailed analysis.

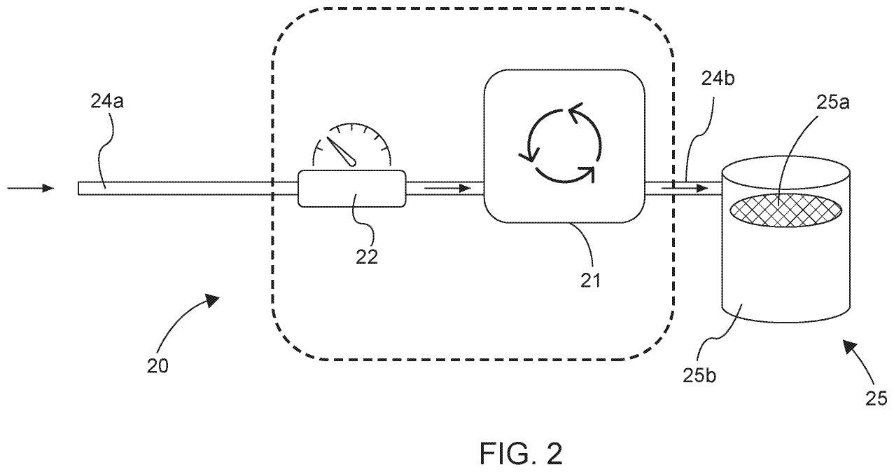

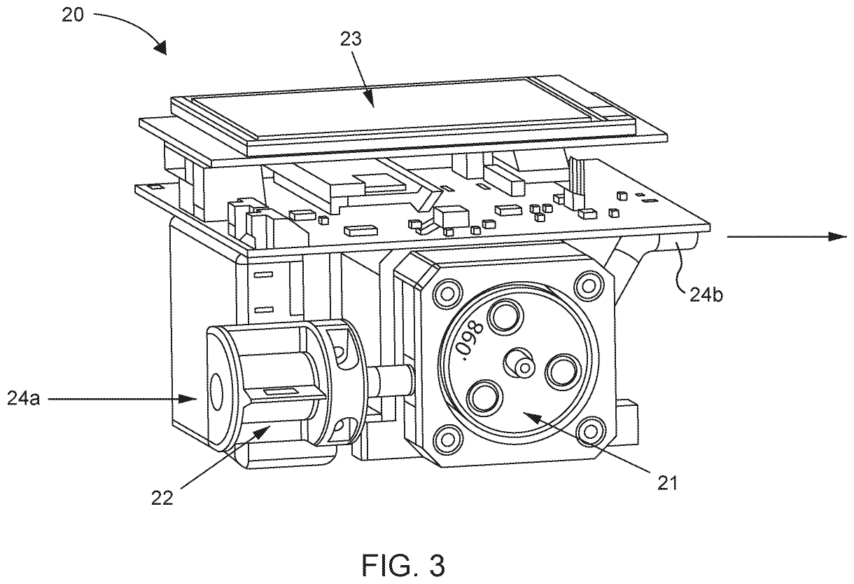

Rather than providing a clot catcher as an integrated portion of a pump system housing as module of pump system 10 according generally to the previously described embodiment of the invention, an alternative embodiment includes a clot catcher provided as an element separate from the pump system, and as depicted schematically in FIG. 2 and as shown in side perspective in FIG. 3.

As shown, pump system 20 comprises a pump 21 and an optional pressure sensor 22. An inlet 24a leading from and communicative with a patient treatment catheter (not shown) is connected to pump system 20. Blood collected via inlet 24a is transferred via an outlet 24b from pump 21 to a separate clot catcher 25 comprising a porous strainer 25a, generally horizontally oriented, through which blood drains into a lower portion of a container/canister 25b (blood collection chamber), retainably depositing any collected clot or clots atop strainer 25a.

The pump system 20 according generally to the depicted embodiment of FIG. 2 is shown in side perspective in FIG. 3. Pump system 20 includes pump 21 configured advantageously in a form which includes a peristaltic pump, the advantageously system 20, is provided with a pressure sensor 22, and optionally further comprises an LCD touch screen for further control of the system. Inlet 23a leading from, and communicative with, a patient catheter (not shown) is connected to outlet 23b from pump 21 and are also identified in FIG. 3.

FIGS. 4A and 4B depict generally the embodiment previously described with reference to FIGS. 1A and 1B, with top housing portion omitted to allow a view of the interior of the integrated clot catcher. In such embodiment, clot catcher 15 is built into a collection compartment 16, of advantageously sterile design. A retrieved clot or clots stay above a perforated strainer provided in the clot catcher 15 co-compartment, while blood collected with the clot(s) drains into the blood collection chamber below, allowing the operator to have the ability within convenient reach and observational scope, the ability to examine the clot(s) and to ascertain a type and composition thereof, as well as the amount of blood withdrawn, to provide therapeutic and diagnostic information.

Since there are well known procedures for determining the compositional nature of various types of clots involved and which are readily understood by those skilled in the art, further discussions of a determination and analysis of such are not discussed further as being deemed unnecessary for purposes herein.

It is noted that while the use of the clot catcher (clot capture device) described herein will most certainly find application in treating human patients, it will be also applicable in veterinary fields of practice, and such use is contemplated to be within the scope of the invention.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention.

What is claimed is:

1. An apparatus, comprising:
a thrombectomy pump;
a housing including a collection chamber and a pump chamber, the thrombectomy pump coupled within the pump chamber;
an aspiration tube extending from the thrombectomy pump within the pump chamber;
a portion of the aspiration tube extending horizontally and in fluid communication with an outlet port in a side wall of the collection chamber such that an aspirated liquid flows from the thrombectomy pump and into the collection chamber; and
a lid removably coupled to a top portion of the housing to cover at least a portion of the collection chamber, the lid including a transparent portion to allow viewing into the collection chamber from a top view.

2. The apparatus of claim 1, further comprising:
a clot retainer within the collection chamber, wherein:
the housing has a base surface, a retention surface of the clot retainer is disposed at a first distance from the base surface of the housing, and
the outlet port in the side wall of the collection chamber is at a second distance from the base surface, the second distance being greater than the first distance.

3. The apparatus of claim 1, wherein:
the lid includes a transparent window that is disposed horizontally above the collection chamber.

4. The apparatus of claim 1, wherein:
the collection chamber surrounds at least a portion of the pump chamber.

5. The apparatus of claim 1, wherein:
the collection chamber has a volume of at least 1000 cubic centimeters.

6. The apparatus of claim 1, wherein the thrombectomy pump is a peristaltic pump.

7. An apparatus, comprising:
a housing including a first housing portion and a second housing portion, the second housing portion defining a collection chamber and a pump chamber integrally formed with the collection chamber, the collection chamber at least partially encircling the pump chamber;
a pump assembly disposed within the pump chamber, the pump assembly including an inlet port configured to be coupled to a catheter and an outlet port in fluid communication with the collection chamber;
the pump assembly including a pump and a controller;
the collection chamber being configured to receive an aspirated liquid flowing from the pump and into the collection chamber; and
a touch screen coupled to the first housing portion and operably coupled to the controller.

8. The apparatus of claim 7, further comprising:
a lid removably couplable to the second housing portion and enclosing at least a portion of the collection chamber.

9. The apparatus of claim 7, wherein:
the collection chamber includes a clot cavity in direct fluid communication with the outlet port.

10. The apparatus of claim 9, further comprising:
a clot retainer having a porous bottom surface to allow liquid to flow through the bottom surface and into the collection chamber.

11. The apparatus of claim 10, wherein:
a lid removably couplable over the clot retainer, the lid including a transparent portion to allow viewing of the clot retainer through the lid.

12. The apparatus of claim 10, wherein:
the housing has a base surface, the outlet port being positioned at a first distance relative to the base surface, the bottom surface of the clot retainer being disposed at a second distance from the base surface, the first distance being greater than the second distance.

13. The apparatus of claim 7, further comprising:
an aspiration tube coupled to the pump within the pump chamber, a portion of the aspiration tube extending horizontally and in fluid communication with the collection chamber.

14. The apparatus of claim 10, further comprising:
an aspiration tube coupled to the pump within the pump chamber, a portion of the aspiration tube extending parallel with the bottom surface of the clot retainer.

15. The apparatus of claim 7, wherein the pump is a peristaltic pump.

16. The apparatus of claim 7, wherein:
the collection chamber has a volume of at least 1000 cubic centimeters.
